(19) United States
(12) Patent Application Publication
(10) Pub. No.: US 2024/0087687 A1
(43) Pub. Date: Mar. 14, 2024

(54) SYSTEMS AND METHODS FOR ONTOLOGY MATCHING

(71) Applicant: Truveta, Inc., Bellevue, WA (US)

(72) Inventors: Cezary Antoni Marcjan, Redmond, WA (US); Mariyam Amir, Lynwood, WA (US); Murchana Baruah, Bothell, WA (US); Mahsa Eslamialishah, Bellevue, WA (US); Sina Ehsani, Seattle, WA (US); Alireza Bahramali, Northampton, MA (US); Sadra Naddaf-Shargh, Mountain View, CA (US); Saman Zarandioon, Seattle, WA (US)

(21) Appl. No.: 18/463,902

(22) Filed: Sep. 8, 2023

Related U.S. Application Data
(60) Provisional application No. 63/375,193, filed on Sep. 9, 2022.
(60) Provisional application No. 63/516,622, filed on Jul. 31, 2023.

(51) Int. Cl. G16B 50/10; G06F 16/31; G16B 50/20

(52) U.S. Cl. CPC G16B 50/10; G06F 16/322; G16B 50/20

(57) ABSTRACT

Systems and methods for aligning ontologies, such as a medical or related ontologies, are disclosed. Initially, ontology specifications are received, such as ontologies comprising a root node and a plurality of child nodes. Each node is assigned at least one synthetic identifier corresponding to its path(s) to the root node. In some cases, nodes may be clustered using one or more clustering algorithms. A translation model is pre-trained by applying one or more masked language models to the ontologies and the synthetic identifiers. Subsequently, each ontology is augmented by identifying nodes in different ontologies that match and assigning label and/or other details across different ontologies. The translation model can then be fine-tuned using the augmented data. The fine-tuned translation model is then used to identify corresponding nodes in target ontologies in response to translation requests.

### CROSS-REFERENCE TO RELATED APPLICATIONS

This application claims the benefit of U.S. Provisional Patent Application No. 63/375,193, filed on Sep. 9, 2022, entitled SYSTEMS AND METHODS FOR DATA NORMALIZATION AND EQUIVALENCE MATCHING BETWEEN ONTOLOGIES and U.S. Provisional Patent Application No. 63/516,622, filed on Jul. 31, 2023, entitled SYSTEMS AND METHODS FOR DATA NORMALIZATION AND EQUIVALENCE MATCHING BETWEEN ONTOLOGIES and is related to U.S. patent application Ser. No. 18/053,654, entitled SYSTEMS AND METHODS FOR DATA NORMALIZATION, filed on Nov. 8, 2022, each of which is herein incorporated by reference in its entirety.

### TECHNICAL FIELD

The present technology generally relates to healthcare, and in particular, to systems and methods for data normalization and ontology matching or ontology alignment.

### BACKGROUND

Medical research has come a long way since paper records were digitized. Researchers now have access to more health data than ever before, but limitations persist. Research is still often conducted on relatively small data sets that may be weeks or even months old and may not represent the full diversity of a population. This can result in biased insights that can compromise patient care. Healthcare entities such as hospitals, clinics, and laboratories produce enormous volumes of health data. This health data can provide valuable insights for research and improving patient care. However, the patient records and other health data received from health system members can arrive from different databases in different formats, ontologies, and so on, often incorporating a wide variety of terminologies, medical code sets, naming conventions, and so on. The structure of these records can also vary widely. Even with standard medical terminology, the way in which that terminology is used can also vary widely. A heart attack may be described as “acute myocardial infarction” in one ontology, “AMI” in another ontology, and so on. Furthermore, each ontology may use a different organizational structure, creating different paths between different concepts in the different ontologies. All of these different structures, terminologies, and semantics can make it difficult to work across health data records and identify meaningful trends and insights. There has been much progress made to arrive at a set of standards and processes that can help address this inconsistency, but the larger and more diverse the dataset, the more complex and time consuming the processing.

### BRIEF DESCRIPTION OF THE DRAWINGS

Many aspects of the present disclosure can be better understood with reference to the following drawings.

FIG. 1 is a block diagram illustrating an environment in which the ontology matching system may operate.

FIG. 2 is a tree diagram illustrating a portion of the SNOMED CT ontology.

FIG. 3 is a tree diagram illustrating a portion of the FMA ontology.

FIG. 4 is a flow diagram illustrating the processing of an ontology matching component.

FIG. 5 is a flow diagram illustrating the processing of a pre-train component.

FIG. 6 is a tree diagram illustrating synthetic hierarchical identifiers for a sample ontology.

FIG. 7 is a flow diagram illustrating the processing of a fine-tune component.

### DETAILED DESCRIPTION

The present technology relates to improved systems and methods for ontology matching or ontology alignment. An ontology provides a set of concepts or categories and the properties and the relations between them. The medical field, for example uses many ontologies to systematically organize a collection of medical terms and provide codes, labels, terms, synonyms, and definitions used in clinical documentation and reporting. For example, an anatomical ontology can be used to define the structure and relationships between features of organisms, such as the various systems of the human body, their individual components, relationships between the components and any of the systems, etc. As another example, a pharmacological ontology can provide information about individual drugs, such as their ingredients, what conditions they are used for, contraindications, their interactions with other drugs, etc. In the medical field, for example, several ontologies, such as The Systemized Nomenclature of Medicine-Clinical Terms (“SNOMED CT” or “SNOMED”), The Foundational Model of Anatomy (“FMA”), the National Cancer Institute (NCI) Thesaurus, etc. are used (wholly or partially) to organize information for various data sources. These ontologies can define concepts and relationships related to, for example, body structures, specimens, organisms and other etiologies, symptoms, diagnoses, procedures, clinical findings, substances, pharmaceuticals, devices, and so on and provide unique identifiers or codes for each element represented in the ontology. However, each ontology may define these concepts and relationships differently and/or use different terminologies, labels, codes, etc.

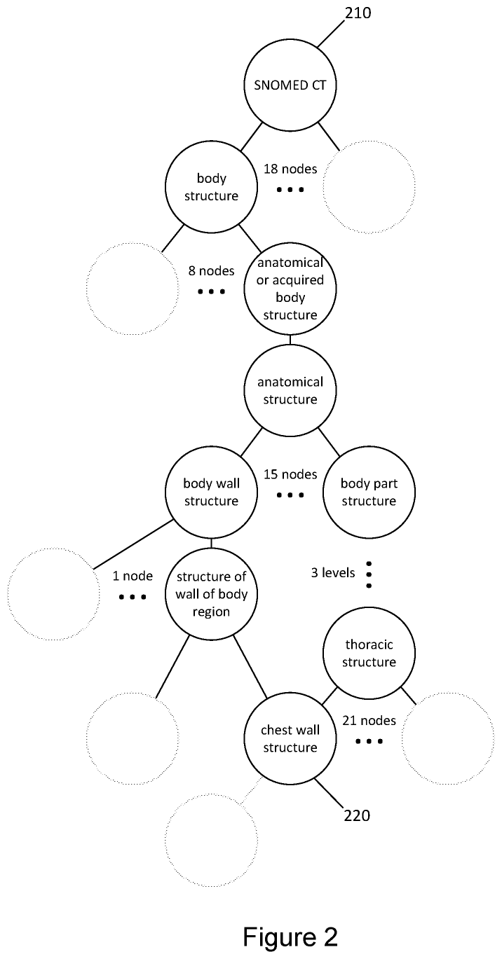

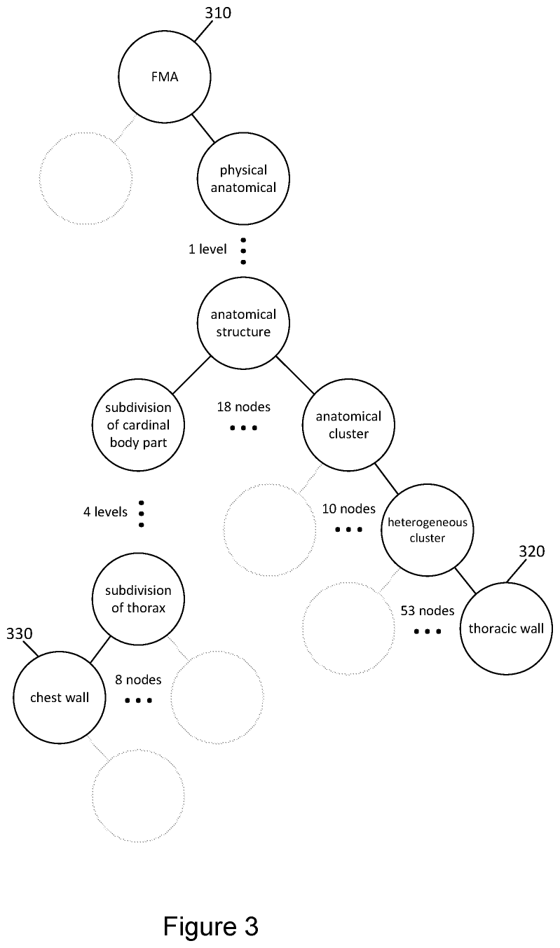

FIG. 2 is a tree diagram illustrating a portion of the SNOMED CT ontology that includes concepts related to chest wall structure. FIG. 3 is a tree diagram illustrating a portion of the FMA ontology that includes concepts similarly related to the chest wall. In this example, the included portions of the ontologies show various paths between the corresponding root nodes 210 and 310 and various nodes in the corresponding ontologies, each node representing a concept, such as “anatomical structure,” “body wall structure,” “body part structure,” “anatomical cluster,” heterogeneous cluster,” etc. Although not every node in each ontology is shown in these examples, one of ordinary skill in the art will recognize that an ontology may comprise any number of nodes, including leaf nodes and non-leaf nodes. For example, the Foundational Model of Anatomy is known to include approximately 75,000 nodes, over 120,000 terms, over 2.1 million relationship instances, over 168 relationship types to link nodes into a coherent symbolic model (sig.biostr.washington.edu/projects/fm/AboutFM.html). Many of the nodes not shown in these examples are collapsed and represented by phrases such as “x nodes,” indicating that x number of nodes are not shown in a corresponding level of ontology, or “x levels” indicating that x number of levels are not shown between two nodes in an ontology, each level including at least one node. In these examples, node 220 in the SNOMED CT ontology (labeled “chest wall structure”), represents the same concepts covered by each of nodes 320 (labeled “thoracic wall”) and 330 (labeled “chest wall”). Accordingly, and as discussed above, different ontologies can represent the same concepts very differently. In this example, the “chest wall structure” concept is covered by a single node in one ontology and multiple nodes in another. Moreover, the paths between each of these nodes can vary in terms of, for example, the length of the path from the root node (i.e., total number of nodes), the labels of the nodes in the path, etc. Thus, if incoming data is coded according to different ontologies, it can be difficult and time consuming to identify relationships between the incoming data even if the incoming data represents the same underlying concepts. Accordingly, an improved process for finding correspondences between these nodes would be greatly beneficial.

Ontology matching or ontology alignment is the process of finding correspondence between the entities (e.g., concepts and relationships) of different ontologies to, for example, unify data from various sources and reduce heterogeneity, making these more viable for research, development, and so on. The correspondence could be associated with element-level matching, structure-level matching, and so on. Because health data platforms interface with health system members throughout the world and billions of clinical data points from this care can be brought together in the health data platform to enable research on any drug, disease, device, etc., it would be helpful to map ontologies quickly and easily in order to unify data from different data sources. Health data platforms can assemble millions of patient records from multiple health provider members. In some embodiments, data flows into the system daily, providing researchers with virtually real time updates. However, the speed, volume, and diversity of this data can pose significant management challenges. For example, the data received from the health system members can include all Electronic Health Record (EHR) data, such as labs, vitals, diagnosis codes, procedure codes, physician notes, imaging reports, pathology reports, images, and/or genomics information. The ontologies and related terminologies associated with these records can vary widely. Accordingly, there is a need for systems and methods that can match or align multiple and disparate ontologies from a large and diverse flow of health data without compromising the diversity and accuracy of that data, or the speed of its delivery for research and/or other purposes.

The inventors have recognized that conventional approaches to ontology matching have significant disadvantages. For example, conventional approaches do not support multi-tasking and use several models to transform between different ontologies. Accordingly, these conventional models are not able to take advantage of transfer learning. As another example, conventional approaches to ontology matching do not learn the overall graph structure of ontologies and rely on direct mappings between individual nodes in different ontologies, without regard to (and the ability to learn from) information in the underlying structures of the ontologies. The disclosed ontology matching system represents an improvement in the technical field of computer-based ontology matching because it is more robust and flexible than current ontology matching systems, can be modified with less time and effort, and reduces the amount of time needed to map ontologies from one to another. Moreover, in some embodiments and in contrast to conventional techniques, the disclosed ontology matching system employs zero-shot learning techniques so that the ontology matching system, via a trained model, can make source-to-target (i.e., source ontology to target ontology) predictions without requiring manually labeled cross-ontologies matching pairs. Furthermore, the disclosed ontology matching system can employ zero-shot prediction techniques to perform mapping to a target ontology (or set of target ontologies) without requiring similarity calculations across the entire source and target ontologies and/or post-processing (e.g., extension/repair).

Accordingly, the inventors have conceived a software and/or hardware ontology matching system for mapping between elements in multiple ontologies. In some embodiments, the ontology matching system initially receives specifications for each of a plurality of ontologies (or sub-ontologies), each ontology defining related concepts and/or categories and relationships between these concepts and/or categories. For example, the ontology matching system may receive an ontology comprising a root node and a number of leaf and non-leaf nodes, each node corresponding to a concept or element in the ontology and connected to one or more other nodes via an edge that defines a relationship between the connected nodes. Moreover, each of the nodes can include a label that describes the related concept or category. For example, in ontologies that define diagnoses, nodes may include labels such as “acute kidney injury,” “acute-on-chronic renal failure,” and so on. As another example, anatomical ontologies nodes may include labels such as “structure of permanent maxillary right second molar tooth,” “structure of permanent mandibular right first molar tooth,” and so on. In some cases, an ontology may include synonyms for one or more labels. For example, a node labeled “Myocardial infarction (disorder)” in the SNOMED CT ontology includes several synonyms, such as “Cardiac infarction,” “Heart attack,” “Infarction of heart,” “MI—myocardial infarction,” and “Myocardial infarct.”

In some embodiments, after the ontology specifications are received, the ontology matching system generates one or more individual synthetic hierarchical identifiers for each individual child node based on paths from a root node of the ontology to the individual child node. In some examples, a root node is assigned the synthetic hierarchical identifier “0” and each synthetic hierarchical identifier for an individual child node is created by appending 1) a separator character (or set of characters), such as “-” and 2) a numerical value to a synthetic hierarchical identifier of the individual child node's parent node. For example, if a root node has three child nodes the child nodes could be assigned synthetic hierarchical identifiers “0-0,” “0-1,” “0-2,” the first “0” in each synthetic hierarchical identifier corresponding to the root node's synthetic hierarchical identifier (i.e., “0”) and the second (or last) numerical value corresponding to each of the different individual nodes. Similarly, individual child nodes of the node assigned synthetic hierarchical identifier “0-0” could be assigned synthetic hierarchical identifiers “0-0-0,” “0-0-1,” “0-0-2,” . . . “0-0-n,” and so on. In this manner, each individual child node is assigned at least one unique synthetic hierarchical identifier. In some cases, there may be multiple paths from the root node to an individual child node. In these cases, the individual child node can be assigned multiple synthetic hierarchical identifiers, each synthetic hierarchical identifier corresponding to a different path from the root node to the individual node. For one example, a node may be assigned synthetic hierarchical identifiers “0-3-5-17-2-23” and “0-2-3-0-9,” each synthetic hierarchical identifier representing a different path from the root node (indicated by the leading “0”) and the individual child node. In this example, “0-3-5-17-2-23” represents a path comprising four nodes between the root node and the individual node and “0-2-3-0-9” represents a path comprising three nodes between the root node and the individual node. In some embodiments, the ontology matching system may designate one of these synthetic hierarchical identifiers as a “primary” synthetic hierarchical identifier and the remaining synthetic hierarchical identifiers as “secondary” or synonym synthetic hierarchical identifiers. For example, the ontology matching system may determine the primary synthetic hierarchical identifier based on the path length associated with each synthetic hierarchical identifier, such as the shortest path. Accordingly, in the above example, the synthetic hierarchical identifier “0-2-3-0-9” would be assigned as the primary synthetic hierarchical identifier for the individual child node and the synthetic hierarchical identifier “0-3-5-17-2-23” would be assigned as a secondary synthetic hierarchical identifier because “0-2-3-0-9” represents a shorter path between the root node and the individual node relative to “0-3-5-17-2-23.”

In some embodiments, rather than relying on the hierarchy provided by an ontology itself, the ontology matching system may generate a semantics-based synthetic hierarchy for an ontology. The ontology matching system can generate embedding vectors for each node in each ontology by, for example, applying the pre-trained model to each node (e.g., to label or synonym data associated with each node) to generate corresponding hierarchical identifiers associated with the different ontologies. In some cases, rather than generating embedding vectors, the ontology matching system relies on the label data included with each ontology or other underlying information that conveys the context of each node. Subsequently, the ontology matching system applies a clustering algorithm to cluster each ontology into a predetermined number of classes (e.g., 32, 64, 100, 128) based on the generated embedding vectors or labels of each node. One of ordinary skill in the art will recognize that any number of clustering algorithms may be applied, such as naive k-means clustering, density-based spatial clustering of applications with noise clustering, and so on. Moreover, the clustering algorithm can be performed recursively on each cluster until the number of concepts in each cluster is reduced to one. Thus, the clustering algorithm can be applied individually to one or more sub-ontologies of an ontology. The ontology matching system also assigns a synthetic semantic identifier to each cluster based on a synthetic semantic identifier assigned to its parent cluster. For example, if the root node is assigned synthetic semantic identifier “0” and a first round of clustering split the ontology into four clusters, those clusters could be assigned synthetic semantic identifiers “0-0,” “0-1,” “0-2,” and “0-3.” Accordingly, the semantics-based synthetic hierarchy represents the ontology as a root node, a number of cluster and/or sub-cluster nodes, each corresponding to a cluster generated by the clustering process, and a number of leaf nodes, each corresponding to a node in the original ontology. In addition to clustering the nodes, the ontology matching system also generates a synthetic semantic identifier for each node based on the synthetic semantic identifier of the cluster to which it belongs. For example, if a cluster assigned synthetic semantic identifier “0-1-5” includes three leaf nodes, those nodes could be assigned synthetic semantic identifiers “0-1-5-0,” “0-1-5-1,” and “0-1-5-2.” The synthetic hierarchy can improve performance of the ontology matching system by reducing path length in an ontology, thereby reducing training time and prediction time when compared to conventional approaches or adding context to relatively flat ontologies.

In some embodiments, after synthetic hierarchical identifiers and/or synthetic semantic identifiers are assigned to the nodes, the ontology matching system begins pre-training a translation model, such as a multi-task sequence-to-sequence transformer model, to learn the structure of each ontology and the semantics of each node in the ontology. In some examples, pre-training is performed by applying one or more trained masked language models to various aspects of each ontology, such as the ontology's labels, synonyms, primary synthetic hierarchical identifiers, secondary synthetic hierarchical identifiers, synthetic semantic identifiers, and so on. Masked language modeling is used to predict a masked value in a sequence of values, such as tokens, words, bytes, etc. A masked language model is a language model trained to predict missing elements based on the context provided by surrounding elements and is done by masking some of the elements in an input sequence and training the model to predict the masked elements based on the context of the non-masked words. In some examples, a masked language model can attend to these values bidirectionally, which means the model has access to sequence values on either side of a masked value. Masked language modeling is useful for tasks where it is helpful to have a contextual understanding of an entire sequence or structure. One of ordinary skill in the art will recognize that any of a number of masked language models can be applied to the ontologies to learn the structure of an ontology, such as the structure of each ontology and the sequences represented by various paths to various nodes in each ontology. For example, “BERT: Pre-training of Deep Bidirectional Transformers for Language Understanding” by Devlin (2019), which is herein incorporated by reference in its entirety, describes a masked language model that randomly masks some values from an input with the objective to predict the original vocabulary id of the masked value based only on its context. As another example, “ByT5. Towards a Token-Free Future with Pre-trained Byte-to-Byte Models” by Xue (Mar. 8, 2022), which is herein incorporated by reference in its entirety, describes a token-free model that operates directly on raw text (bytes or characters). In some examples, the ontology matching system employs a multi-task model, which enables the model to implicitly learn the relationship between different ontologies via transfer-learning, without requiring any explicit cross-ontology manually labeled data. This also enables the disclosed ontology matching system to outperform existing solutions both from runtime complexity and quality of alignments. In some embodiments, the model is improved over a series of training epochs by gradually increasing the masking percentage (e.g., 10%, 15%, 20%, 30%, 45%) over each training epoch. In this manner, the ontology matching system improves the accuracy of the model by repeatedly re-training the translation model to further adjust the model's parameters.

In some embodiments, the ontology matching system augments each of the ontologies by identifying matches present in the labels and synonyms of the other ontologies. For example, the ontology matching system may identify each node label and any corresponding synonyms in one ontology and, for each identified node label or synonym, search each of the other ontologies to find nodes with matching labels and/or synonyms. In some cases, the ontology matching system may require an exact match between elements (labels and/or synonyms). In some examples, the ontology matching system may use a string metric, such as Levenshtein distance or other edit distances, to determine whether the difference between to elements (labels and/or synonyms) is below a predetermined threshold (considered a match). If a match is found, information identified with the matching elements can be added to each of the corresponding nodes, such as labels, node identifiers, synonyms, and so on. In some cases, the ontology matching system may augment each ontology based on matches found in a medical dictionary or thesaurus, such as the UNIFIED MEDICAL LANGUAGE SYSTEM (“UMLS”), which serves as a thesaurus and ontology of biomedical concepts, providing vocabularies in the biomedical sciences and maps structures among these vocabularies. This ontology augmentation process expands the training corpus, enriches the data by adding cross-ontology information with minimal processing, and helps to perform more comprehensive learning.

In some embodiments, after the ontologies have been augmented, the ontology matching system further adjusts the translation model by training the translation model using the augmented ontologies to fine-tune or improve the model parameters based on the enriched data offered by the expanded training corpus. In some examples, this process is performed on the augmented ontologies in a multi-task manner, using sequence-to-sequence translation from a label/synonym to a synthetic identifier, which improves the model's ability to learn/identify a node in a target ontology in the form of a synthetic identifier (hierarchical or semantic) associated with the node, given an input string. In this manner, the translation model is trained to identify a synthetic identifier (hierarchical or semantic) associated with a target ontology based on an input string, such as an input string provided by a user, a label associated with a node in a source ontology (i.e., an ontology being mapped to a target ontology), and so on.

In some embodiments, the ontology matching system stores data about one or more patients in a standardized format in a plurality of network-based computer-readable storage devices having a collection of medical records stored thereon and provides access to remote users over a network so that the users can modify the data in real time, wherein at least one of the users provides modified data in a non-standardized format dependent on the hardware and software platform used by the at least one of the users. For example, the non-standardized format may include the use of a particular ontology (and its labels) associated with the software platform used by the least one user. In this case, the ontology matching system can convert the non-standardized modified data into a standardized format by, for example, applying the trained translation model to labels associated with the software platform's ontology and included in the modified data to identify corresponding nodes (and labels) in a preferred target ontology. In this manner, the modified data can be standardized to the preferred, target ontology (e.g., by adding the corresponding labels to the modified data) and stored in the collection of medical records in the standardized format. Moreover, the ontology matching system can automatically generate a message containing the modified data when the modified data has been stored transmit the message to all of the users over the computer network in real time, so that each user has immediate access to up-to-date medical records.

In some embodiments, the ontology matching system receives or collects a set of ontologies and applies one or more transformations to each ontology, such as masking one or more elements of each ontology to create a modified set of ontologies. The ontology matching system can use this modified set of ontologies and the collected set of ontologies to create a first training set and then train a translation model in a first stage using the first training set. Subsequently, the ontology matching system can augment the ontologies by identifying matching labels between nodes of two or more ontologies and adding corresponding labels and/or synonyms to the matching nodes. The ontology matching system can then create a second training set that includes the first training set and the augmented ontologies and train the translation model in a second stage using the second training set.

The above-mentioned processes improve the field of ontology matching by providing a more robust and flexible ontology matching system that can be modified with less time and effort, and that reduces the amount of time needed for ontology alignment. These improvements result from specific software components described herein, and thus are reasonably interpreted as technical improvements, not as improvements in a method of organizing human activity.

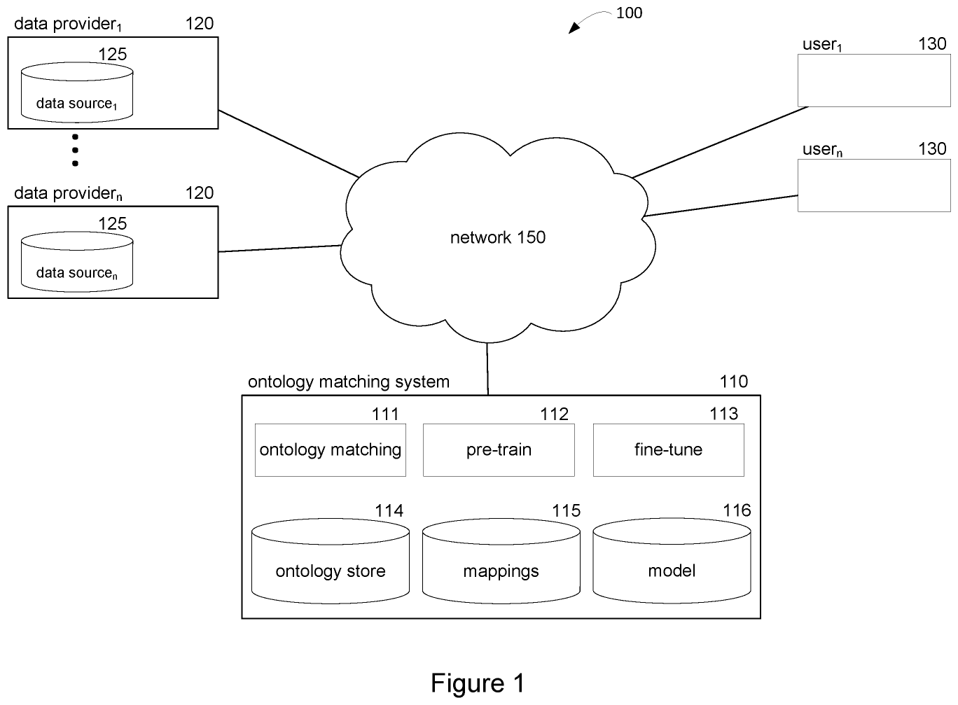

FIG. 1 is a block diagram illustrating an environment 100 in which the ontology matching system may operate in accordance with some embodiments of the disclosed technology. In this example, environment 100 comprises ontology matching system 110, data provider computing systems 120, and user computer systems 130. Ontology matching system 110 comprises ontology matching component 111, pre-train component 112, fine-tune component 113, ontology store 114, mappings store 115, and model store 116. Ontology matching system 110 invokes ontology matching component 111 to receive ontology data and align ontologies by pre-training and fine-tuning a translation model, such as a multi-task sequence-to-sequence transformer model, using language modeling techniques, such as masked language modeling techniques, etc. Ontology matching component 111 invokes pre-train component 112 to pre-train the translation model and invokes fine-tune component 113 to fine-tune the translation model. In some examples, the ontology matching system may periodically (e.g., daily, weekly, monthly, etc.) update the translation model to ensure that it is up to date and based on the most current ontology information. Ontology store 114 stores information about ontologies received from one or more data providers and ontology information generated by the ontology matching system, such as synthetic hierarchical identifiers, synthetic semantic identifiers, augmented ontology data, and so on. In some examples, the ontology matching system can download and store ontologies periodically (e.g., weekly, monthly, annually, etc., or when an updated ontology is released). In some cases, the ontology store stores multiple versions of one or more ontologies, such as current and previously released versions. Mappings store 115 stores mappings between nodes in one ontology and nodes in another ontology, such as mappings generated by the ontology matching system. Model store 116 stores information about ontology translation models, such as the model itself, model parameters, an indication of the ontologies used to train the model, when the model was trained, and so on. Data providers, such as ontology providers, healthcare systems, research institutions, etc. can interact with the ontology matching system 110 via data provider computing systems 120 over network 150 using a user interface provided by, for example, an operating system, web browser, listings viewer application, or other application. Similarly, users, such as medical professionals, researchers, developers, and so on can interact with the ontology matching system 110 via user computing systems 130 over network 150 using a user interface provided by, for example, an operating system, web browser, listings viewer application, or other application.

The computing devices and systems on which the ontology matching system can be implemented can include a central processing unit, input devices, output devices (e.g., display devices and speakers), storage devices (e.g., memory and disk drives), network interfaces, graphics processing units, accelerometers, cellular radio link interfaces, global positioning system devices, and so on. The input devices can include keyboards, pointing devices, touchscreens, gesture recognition devices (e.g., for air gestures), thermostats, smart devices, head and eye tracking devices, microphones for voice or speech recognition, and so on. The computing devices can include desktop computers, laptops, tablets, e-readers, personal digital assistants, smartphones, gaming devices, servers, and computer systems such as massively parallel systems. The computing devices can each act as a server or client to other server or client devices. The computing devices can access computer-readable media that includes computer-readable storage media and data transmission media. The computer-readable storage media are tangible storage means that do not include transitory, propagating signals. Examples of computer-readable storage media include memory such as primary memory, cache memory, and secondary memory (e.g., CD, DVD, Blu-Ray) and include other storage means. Moreover, data may be stored in any of a number of data structures and data stores, such as a databases, files, lists, emails, distributed data stores, storage clouds, etc. The computer-readable storage media can have recorded upon or can be encoded with computer-executable instructions or logic that implements the ontology matching system, such as a component comprising computer-executable instructions stored in one or more memories for execution by one or more processors. In addition, the stored information can be encrypted. The data transmission media are used for transmitting data via transitory, propagating signals or carrier waves (e.g., electromagnetism) via a wired or wireless connection. In addition, the transmitted information can be encrypted. In some cases, the ontology matching system can transmit various alerts to a user based on a transmission schedule, such as an alert to inform the user that an update to an ontology has prompted the ontology matching system to update one or more translation models for performing ontology matching. Furthermore, the ontology matching system can transmit an alert over a wireless communication channel to a wireless device associated with a remote user or a computer of the remote user based upon a destination address associated with the user and a transmission schedule in order to, for example, periodically recommend invoking the ontology matching component to update an ontology translation model. In some cases, such an alert can activate an application to cause the alert to display, on a remote user computer and to enable a connection via, a universal resource locator (URL), to a data source over the internet, for example, when the wireless device is locally connected to the remote user computer and the remote user computer comes online. Various communications links can be used, such as the Internet, a local area network, a wide area network, a point-to-point dial-up connection, a cell phone network, and so on for connecting the computing systems and devices to other computing systems and devices to send and/or receive data, such as via the Internet or another network and its networking hardware, such as switches, routers, repeaters, electrical cables and optical fibers, light emitters and receivers, radio transmitters and receivers, and the like. While computing systems and devices configured as described above are typically used to support the operation of the ontology matching system, those skilled in the art will appreciate that the ontology matching system can be implemented using devices of various types and configurations, and having various components.

The ontology matching system can be described in the general context of computer-executable instructions, such as program modules and components, executed by one or more computers, processors, or other devices, including single-board computers and on-demand cloud computing platforms. Generally, program modules or components include routines, programs, objects, data structures, and so on that perform particular tasks or implement particular data types. Typically, the functionality of the program modules can be combined or distributed as desired in various embodiments. Aspects of the ontology matching system can be implemented in hardware using, for example, an application-specific integrated circuit (“ASIC”).

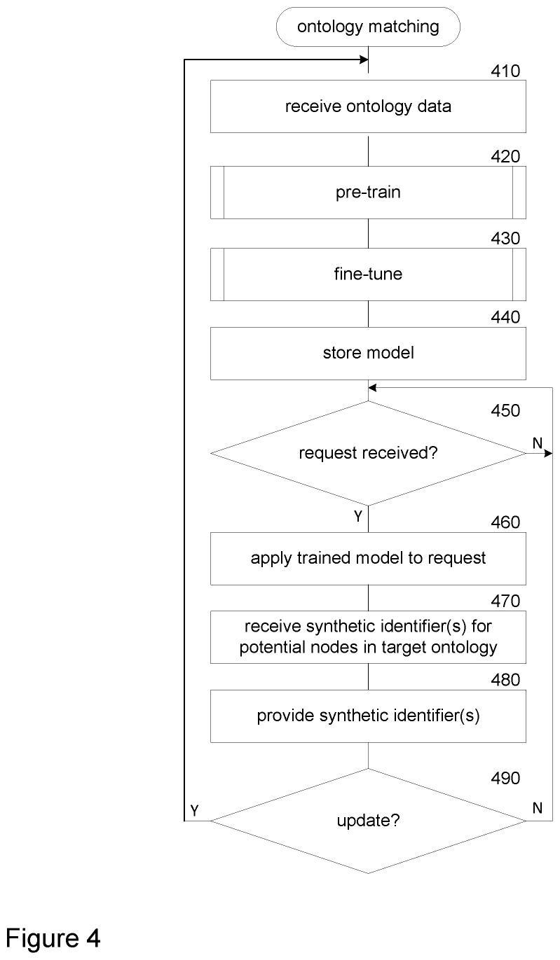

FIG. 4 is a flow diagram illustrating the processing of an ontology matching component in accordance with some embodiments of the disclosed technology. The ontology matching system invokes the ontology matching component to pre-train and fine-tune a translation model for translating between multiple ontologies and apply the translation model to the translation request. In block 410, the ontology matching component receives ontology data, such as ontology data downloaded from one or more publicly or privately available ontology developers or owners, such as SNOMED CT from International Health Terminology Standards Development Organization, the Foundational Model of Anatomy from the Structural Informatics Group at the University of Washington, and so on. The ontologies (or sub-ontologies) to which the ontology matching component is applied may be designated by a user or determined automatically based on, for example, available ontology data, gaps in ontology translation data, and so on. In block 420, the ontology matching component invokes a pre-train component to pre-train the translation model based on the received ontology data. In block 430, the component invokes a fine-tune component to further adjust the model parameters of the translation model. In block 440, the ontology matching component stores the fine-tune model in, for example, a model store. In decision block 450, if a request to translate (or translation request) a label to a node (e.g., a node identifier) in a target ontology is received, then the ontology matching component continues at block 460, else the ontology matching component continues to wait for a request. In some examples, the ontology component may cease processing until a request is received. In some examples, if a request is not received within a predetermined period (e.g., day, week, month) the component may proceed (not shown) to block 485. In some examples, the request may include a string of text and a target ontology (or set of target ontologies), such as (“chest area” and “SNOMED CT”) or (“heart attack” and “FMA and SNOMED CT”). In some cases, the request may not identify a target ontology, in which case the ontology matching system may generate outputs (i.e., node identifiers) for all ontologies for which the model has been trained. In some cases, the ontology matching system may receive a request from a user or a request from an automated system, such as a system configured to perform a complete or partial translation of one ontology (“the source ontology”) to another ontology (“the target ontology”) by sending, to the ontology matching system, multiple translation requests, each request including a label for a corresponding node and an indication of the target ontology. In some cases, the ontology matching system may store mappings between nodes in different ontologies in a mappings store. For example, if the translation model were to map a label associated with a node assigned the synthetic hierarchical identifier “0-3-3-4-4-3-3” in the SNOMED CT ontology to the node assigned the synthetic hierarchical identifier 0-1-4-6-3-3” in the FMA ontology, the ontology matching system may store an indication of this mapping (e.g., “SNOMED CT|0-3-3-4-4-3-3|hierarchical”===>“FMA|0-2-3-3|hierarchical”) in a mappings store for future reference. One of ordinary skill in the art will recognize that additional details about this mapping may also be stored in the mapping store, such as the time the mapping was made, version information for the ontologies, used to generate the mapping, and so on. In block 470, the ontology matching component receives an indication of the identified nodes from the trained model, such as one or more synthetic identifiers (e.g., synthetic hierarchical identifiers or synthetic semantic identifiers). In block 480, the component provides the received indications (e.g., synthetic identifiers) to the requesting party. In decision block 490, if the ontology matching component identifies a reason for updating the translation model, then the ontology matching component loops back to block 410 to receive any updated ontology data and re-train the translation model, else the ontology matching component loops back to decision block 450 to wait for another translation request. For example, the ontology matching component may update the translation model periodically or in response to determining that any underlying ontology data has been updated.

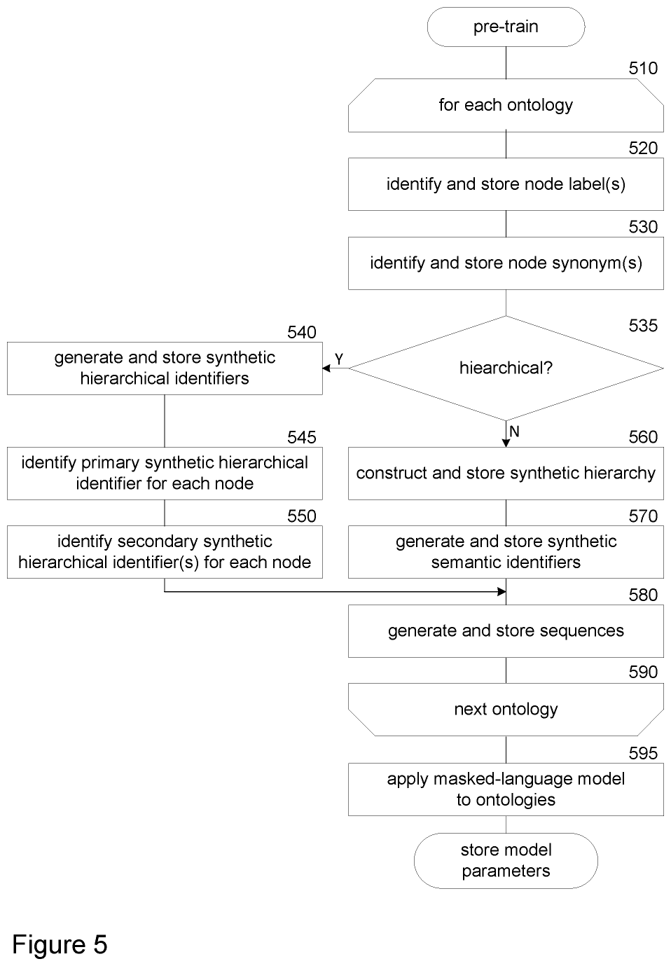

FIG. 5 is a flow diagram illustrating the processing of a pre-train component in accordance with some embodiments of the disclosed technology. In blocks 510-590, the pre-train component loops through each of a plurality of specified ontologies (or sub-ontologies) to pre-train a translation model for translating text, such as plain text provided by a user or label associated with a node in a “source ontology” to a node in a “target ontology,” by generating various paths or sequences for each node, such as a sequence based on labels of nodes, a sequence based on synthetic identifiers, a sequence based on synonyms, and so on. In this manner, a dataset (i.e., labels, identifiers, synonyms) in a particular path between the root node and the node represents a sequence of corresponding values (e.g., a sequence of labels, a sequence of identifiers, a sequence of synonyms, etc.). Thus, multiple sequences can be generated for a path in an ontology. In block 520, the pre-train component identifies a label for each node in the ontology based on the received ontology data. In block 530, the pre-train component identifies any synonyms for each node in the ontology based on the received ontology data. In some cases, a node in an ontology may not include any synonym data. In decision block 535, if the pre-train component is configured to pre-train the translation model according to the hierarchy of the underlying ontology, then the pre-train component continues at block 540, else the component continues at block 560 to construct a synthetic hierarchy. For example, the option to train the translation model based on the underlying hierarchies may be determined at run-time by a user or pre-configured based on one or more design preferences. For example, if one or more of the ontologies is relatively flat (e.g., has fewer than a predetermined number of levels, such as two or three) or has a height that exceeds a predetermined value (e.g., 5, 10, 20), then the pre-train component may opt to construct a synthetic hierarchy for the ontologies. In block 54s0, the pre-train component generates one or more synthetic hierarchical identifiers for each node in the hierarchy based on each path between the root node and the node.

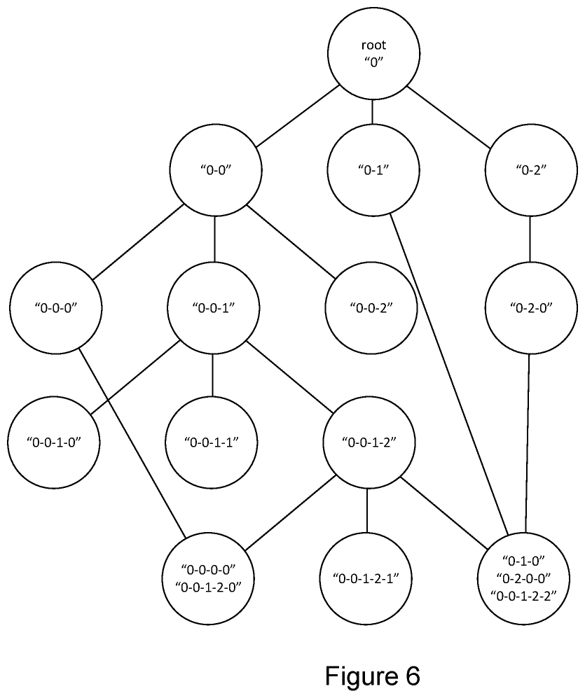

FIG. 6 is a tree diagram illustrating synthetic hierarchical identifiers for a sample ontology in accordance with some embodiments of the disclosed technology. In this example, each node is represented by one or more synthetic identifiers, based on the number of paths from the root node to the node. In this example, the root node is assigned a synthetic identifier of “0.” Each of its child nodes are assigned a synthetic identifier that is based on the root node's synthetic hierarchical identifier (i.e., “0”) and another value, starting with “0.” Thus, the child nodes of the root node in this example are assigned synthetic hierarchical identifiers “0-0,” “0-1,” and “0-2.” The child nodes of these nodes are assigned synthetic hierarchical identifiers based on their parent node's synthetical hierarchical identifier. Thus, the child nodes of “0-0” are assigned synthetic hierarchical identifiers “0-0-0,” “0-0-1,” “0-0-2.” As illustrated, some nodes may be assigned multiple synthetic hierarchical identifiers if there are multiple paths to the node. For example, there are three paths to the node with the synthetic hierarchical identifiers “0-1-0.” Thus, in addition to “0-1-0,” that node is also assigned synthetic hierarchical identifiers “0-2-0-0” and “0-0-1-2-2.” Similarly, the node assigned synthetic hierarchical identifier “0-0-0-0” is also assigned synthetic hierarchical identifier “0-0-1-2-0.” One of ordinary skill in the art will recognize that any number of separator values and/or counter values may be used to generate synthetic identifiers. For example, rather than using “-” as separate values, the ontology matching system may use “+,” “*,” “->,” and so on. Similarly, rather than using a numerical counter that starts at 0 for each level, the ontology matching system may use letters (e.g., a-a-b-a) or a counter that starts at a value other than 0 (e.g., 1-1-2-1), and so on.

Returning to FIG. 5, at block 545, the pre-train component identifies a primary synthetic hierarchical identifier for each node based on the length of each synthetic hierarchical identifier assigned to a node. In some examples, the synthetic hierarchical identifier with the shortest length is designated as the primary synthetic hierarchical identifier for that node. In block 550, the pre-train component designates any other synthetic hierarchical identifiers assigned to a node as secondary synthetic hierarchical identifiers. In block 560, rather than generate synthetic hierarchical identifiers for the ontology, the component constructs a synthetic hierarchy for the ontology. As discussed above, the pre-train component can construct a synthetic hierarchy by applying a clustering algorithm, such as k-means clustering, to the nodes of the ontology to split the ontology into a number of clusters. The clustering algorithm can then be applied to each cluster to further split the cluster into smaller and smaller clusters until the number of concepts in each cluster is reduced to one. In this manner, the pre-train component creates a synthetic structure comprising a root node, a number of non-leaf nodes each corresponding to a concept or a group of concepts (e.g., concepts identified by the clustering algorithm), and a number of leaf nodes, each corresponding to nodes from the underlying ontology. In some examples, the pre-train component may apply a metric (e.g., silhouette coefficient, Dunn index, simple matching coefficient (SMC), etc.) to the generated clusters to determine the quality of the clusters. If the quality (e.g., average (mean, median, mode) value of the metric(s)) is below a predetermined threshold, the pre-train component may attempt to re-cluster the nodes by adjusting one or more parameters of the clustering algorithm or using a different clustering algorithm. In block 570, the pre-train component generates synthetic semantic identifiers for each node in the synthetic hierarchy, as discussed above with respect to synthetic hierarchical identifiers, the synthetic semantic identifiers can be generated by assigning “0” to the root node and appending values at each level of the synthetic hierarchy. In block 580, the component generates and stores sequences for the ontology based on the labels, synonyms, synthetic identifiers, and so on, each sequence corresponding to path of nodes and an attribute of each node. For example, the pre-train component can generate “label sequences” by concatenating node labels and separator values to construct a sequence of labels for each node, each sequence representing a list of labels from the root node to the individual node. Accordingly, each path in the ontology can be converted into one or more sequences based on the underlying and generated/collected data of the ontology. In block 590, if there are any ontologies left to process, then the component loops back to block 510 to select the next ontology, else the component continues at block 595. In block 595, the pre-train component applies a trained masked-language model to the ontologies and their corresponding sequences to generate parameters for the translation model. In some embodiments, the masked language model is a multi-task sequence-to-sequence transformer model, which enables the model to implicitly learn relationship between different ontologies and their corresponding sequences via transfer-learning.

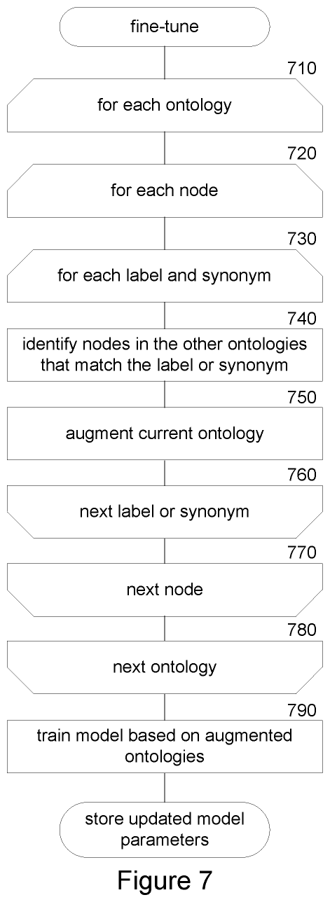

FIG. 7 is a flow diagram illustrating the processing of a fine-tune component in accordance with some embodiments of the disclosed technology. In blocks 710-780, the fine-tune component loops through each of the ontologies to augment data associated with those ontologies. In blocks 720-770, the fine-tune component loops through each node of the currently-selected ontology to augment data associated with each node. In blocks 730-760, the fine-tune component loops through each label or synonym of the currently-selected node. In block 740, the fine-tune component identifies nodes in the other ontologies (i.e., the ontologies other than the currently-selected ontology) that have labels or synonyms that match the currently-selected label or synonym, such as an exact match or a match that is within an edit metric or distance. In block 750, the fine-tune component augments the currently-selected node by adding information associated with the identified nodes to the currently-selected node, such as label data, synonym data, a synthetic identifier associated with the node, and so on. In block 760, if there are any labels or synonyms of the currently-selected node left to process, then the fine-tune component loops back to block 730 to process the next label or synonym, else the fine-tune component continues at block 770. In block 770, if there are any nodes of the currently-selected ontology left to process, then the fine-tune component loops back to block 720 to process the next node, else the fine-tune component continues at block 780. In block 780, if there are any ontologies left to process, then the fine-tune component loops back to block 710 to process the next ontology, else the fine-tune component continues at block 790. In block 790, the fine-tune component further trains the translation model using the augmented ontologies and their corresponding sequences to update weights and parameters of the translation model and then stores the updated model parameters. One of ordinary skill in the art will recognize that in some cases, the fine-tuning may be applied to all of the translation model's weights and parameters. In some cases, one or more weights or parameters may be frozen (i.e., not updated during the fine-tuning process). Moreover, the translation model can be fine-tuned by using the pre-trained model parameters and adding one or more task-specific layers trained using the underlying and/or augmented data.

Unless the context clearly requires otherwise, throughout the description and the claims, the words “comprising,” “comprise,” and the like are to be construed in an inclusive sense, as opposed to an exclusive or exhaustive sense; that is to say, in the sense of “including, but not limited to.” As used herein, the terms “coupled,” “connected,” or any variant thereof, mean any connection or coupling, either direct or indirect, between two or more elements; the coupling or connection between the elements can be physical, logical, or a combination thereof. Additionally, the words “herein,” “above,” “below,” and words of similar import, when used in this application, refer to this application as a whole and not to any particular portions of this application. Where the context permits, words in the above Detailed Description using the singular or plural number can also include the plural or singular number, respectively. The word “or” in reference to a list of two or more items, covers all of the following interpretations of the word: any of the items in the list all of the items in the list, and any combination of the items in the list.

The above Detailed Description of examples of the disclosed subject matter is not intended to be exhaustive or to limit the disclosed subject matter to the precise form disclosed above. While specific examples for the disclosed subject matter are described above for illustrative purposes, various equivalent modifications are possible within the scope of the disclosed subject matter, as those skilled in the relevant art will recognize. For example, while processes or blocks are presented in a given order, alternative implementations can perform routines having steps, or employ systems having blocks, in a different order, and some processes or blocks can be deleted, moved, added, subdivided, combined, and/or modified to provide alternative combinations or sub-combinations. Each of these processes or blocks can be implemented in a variety of different ways. Also, while processes or blocks are at times shown as being performed in series, these processes or blocks can instead be performed or implemented in parallel, or can be performed at different times. Further, any specific numbers noted herein are only examples: alternative implementations can employ differing values or ranges.

The disclosure provided herein can be applied to other systems, and is not limited to the system described herein. The features and acts of various examples included herein can be combined to provide further implementations of the disclosed subject matter. Some alternative implementations of the disclosed subject matter can include not only additional elements to those implementations noted above, but also can include fewer elements.

Any patents and applications and other references noted herein, including any that can be listed in accompanying filing papers, are incorporated herein by reference in their entireties. Aspects of the disclosed subject matter can be changed, if necessary, to employ the systems, functions, components, and concepts of the various references described herein to provide yet further implementations of the disclosed subject matter.

These and other changes can be made in light of the above Detailed Description. While the above disclosure includes certain examples of the disclosed subject matter, along with the best mode contemplated, the disclosed subject matter can be practiced in any number of ways. Details of the ontology matching system can vary considerably in the specific implementation, while still being encompassed by this disclosure. Terminology used when describing certain features or aspects of the disclosed subject matter does not imply that the terminology is being redefined herein to be restricted to any specific characteristics, features, or aspects of the disclosed subject matter with which that terminology is associated. In general, the terms used in the following claims should not be construed to limit the disclosed subject matter to specific examples disclosed herein, unless the above Detailed Description section explicitly defines such terms. The scope of the disclosed subject matter encompasses not only the disclosed examples, but also all equivalent ways of practicing or implementing the disclosed subject matter under the claims.

To reduce the number of claims, certain aspects of the disclosed subject matter are presented below in certain claim forms, but the applicant contemplates the various aspects of the disclosed subject matter in any number of claim forms. For example, aspects of the disclosed subject matter can be embodied as a means-plus-function claim, or in other forms, such as being embodied in a computer-readable medium. (Any claims intended to be treated under 35 U.S.C. § 112(f) will begin with the words “means for,” but use of the term “for” in any other context is not intended to invoke treatment under 35 U.S.C. § 112(f).) Accordingly, the applicant reserves the right to pursue additional claims after filing this application to pursue such additional claim forms, in either this application or in a continuing application.

Although the subject matter has been described in language specific to structural features and/or methodological acts, it is to be understood that the subject matter defined in the appended claims is not necessarily limited to the specific features or acts described above. The specific features and acts described above are disclosed as example forms of implementing the claims.

From the foregoing, it will be appreciated that specific embodiments of the disclosed subject matter have been described herein for purposes of illustration, but that various modifications can be made without deviating from the scope of the disclosed subject matter. For example, although described as being performed on an entire ontology, one of ordinary skill in the art will understanding that the above-described systems, methods, and processes can be applied to sub-ontologies or other portions of an ontology. In other words, any sub-ontology of an ontology can be considered and treated as its own ontology. Moreover, one of ordinary skill in the art will recognize that the ontologies are provided as an example type of hierarchies, that the disclosed subject matter can be used to match or align hierarchies other than ontologies. Additionally, while advantages associated with certain embodiments of the new technology have been described in the context of those embodiments, other embodiments can also exhibit such advantages, and not all embodiments need necessarily exhibit such advantages to fall within the scope of the technology. Accordingly, the disclosure and associated technology can encompass other embodiments not expressly shown or described herein. Although the subject matter has been described in language specific to structural features and/or methodological acts, it is to be understood that the disclosed subject matter is not necessarily limited to the specific features or acts described above. Rather, the specific features and acts described above are disclosed as example forms of the disclosed subject matter. To the extent any materials incorporated herein by reference conflict with the present disclosure, the present disclosure controls.

The present technology is illustrated, for example, according to various aspects described below. Various examples of aspects of the present technology are described as numbered examples for convenience. These are provided as examples and do not limit the present technology. It is noted that any of the dependent examples may be combined in any combination, and placed into a respective independent example. The other examples can be presented in a similar manner.

Example 1: A method, performed by a computing system having a memory and a processor, for aligning a first ontology of a first data source and a second ontology of a second data source, the method comprising: receiving a specification for the first ontology, the first ontology comprising a first root node and a first plurality of child nodes, each child node having a label; receiving a specification for the second ontology, the second ontology comprising a second root node and a second plurality of child nodes, each child node having a label; for each child node of the first ontology, assigning at least one synthetic identifier to the child node, wherein each synthetic identifier has a length that is based on a path from the first root node to the child node of the first ontology; for each child node of the second ontology, assigning at least one synthetic identifier to the child node, wherein each synthetic identifier has a length that is based on a path from the second root node to the child node of the second ontology; pre-training a translation model at least in part by, applying one or more masked language models to the first ontology, and applying one or more masked language models to the second ontology; for each of a plurality of labels of the first ontology, identifying one or more labels of the second ontology that match the label of the first ontology, augmenting the first ontology based on the identified one or more labels of the second ontology, and augmenting the second ontology based on the label of the first ontology; fine-tuning the pre-trained translation model at least in part by training the translation model on the augmented first ontology and the augmented second ontology; receiving a translation request, the translation request include a text string and identifying a target ontology; and applying the fine-tuned translation model to the received translation request to identify at least one or more child nodes of the target ontology that correspond to the text string of the received translation request.

Example 2: The method of any of the Examples herein, further comprising: for each of a plurality of child nodes of the first ontology, applying the fine-tuned translation model to the child node to identify one or more child nodes of the second ontology, and for each identified one or more child nodes of the second ontology, storing a synthetic identifier assigned to the identified child node of the second ontology in association with the child node of the first ontology.

Example 3: The method of any of the Examples herein, further comprising: for a first child node of the first plurality of child nodes, selecting, from among a plurality of synthetic identifiers assigned to the first child node, a primary synthetic identifier based on the length of each of the plurality of synthetic identifiers assigned to the first child node.

Example 4: The method of any of the Examples herein, wherein applying the one or more masked language models to the first ontology comprises masking a first percentage of labels in a first iteration and masking a second percentage of labels in a second iteration, wherein the second percentage is greater than the first percentage.

Example 5: The method of any of the Examples herein, further comprising: determining a height of the first ontology; and in response to determining that the determined height is greater than a predetermined threshold, applying a clustering algorithm to the first plurality of child nodes to generate a synthetic hierarchy for the first ontology.

Example 6: The method of any of the Examples herein, further comprising: generating synthetic semantic identifiers for each of a plurality of cluster nodes and leaf nodes of the synthetic hierarchy.

Example 7: The method of any of the Examples herein, further comprising: for each identified one or more child nodes of the second ontology, storing a synthetic identifier assigned to the child node of the first ontology in association with the identified child node of the second ontology.

Example 8: A computing system for aligning ontologies, the computing system comprising: at least one processor; at least one memory; a component configured to receive a specification for a first ontology, the first ontology comprising a first root node and a first plurality of child nodes, each child node having a label; a component configured to receive a specification for a second ontology, the second ontology comprising a second root node and a second plurality of child nodes, each child node having a label; a component configured to, for each child node of the first ontology, assign at least one synthetic identifier to the child node, wherein each synthetic identifier has a length that is based on a path from the first root node to the child node of the first ontology; a component configured to, for each child node of the second ontology, assign at least one synthetic identifier to the child node, wherein each synthetic identifier has a length that is based on a path from the second root node to the child node of the second ontology; a component configured to, pre-train a translation model at least in part by, applying one or more masked language models to the first ontology, and applying one or more masked language models to the second ontology; a component configured to, for each of a plurality of labels of the first ontology, identify one or more labels of the second ontology that match the label of the first ontology, augment the first ontology based on the identified one or more labels of the second ontology, and augment the second ontology based on the label of the first ontology; a component configured to fine-tune the pre-trained translation model at least in part by training the translation model on the augmented first ontology and the augmented second ontology; a component configured to receive a translation request, the translation request include a text string and identifying a target ontology; and a component configured to apply the fine-tuned translation model to the received translation request to identify at least one or more child nodes of the target ontology that correspond to the text string of the received translation request, wherein each component comprises computer-executable instructions stored in the at least one memory for execution by the computing system.

Example 9: The computing system of any of the Examples herein, further comprising: a component configured to, for each of a plurality of child nodes of the first ontology, apply the fine-tuned translation model to the child node to identify one or more child nodes of the second ontology, and for each identified one or more child nodes of the second ontology, store a synthetic identifier assigned to the identified child node of the second ontology in association with the child node of the first ontology.

Example 10: The computing system of any of the Examples herein, further comprising: a component configured to, for a first child node of the first plurality of child nodes, select, from among a plurality of synthetic identifiers assigned to the first child node, a primary synthetic identifier based on the length of each of the plurality of synthetic identifiers assigned to the first child node.

Example 11: The computing system of any of the Examples herein, wherein the component configured to apply the one or more masked language models to the first ontology is further configured to mask a first percentage of labels in a first iteration and mask a second percentage of labels in a second iteration, wherein the second percentage is greater than the first percentage.

Example 12: The computing system of any of the Examples herein, further comprising: a component configured to determine a height of the first ontology; and a component configured to, in response to determining that the determined height is greater than a predetermined threshold, apply a clustering algorithm to the first plurality of child nodes to generate a synthetic hierarchy for the first ontology.

Example 13: The computing system of any of the Examples herein, further comprising: a component configured to generate synthetic semantic identifiers for each of a plurality of cluster nodes and leaf nodes of the synthetic hierarchy.

Example 14: The computing system of any of the Examples herein, further comprising: a component configured to, for each identified one or more child nodes of the second ontology, store a synthetic identifier assigned to the child node of the first ontology in association with the identified child node of the second ontology.

Example 15: A computer-readable storage medium storing instructions that, when executed by a computing system having a memory and a processor, cause the computing system to perform a method for aligning a plurality of ontologies, the method comprising: receiving a specification for a first ontology, the first ontology comprising a first root node and a first plurality of child nodes, each child node having a label; receiving a specification for a second ontology, the second ontology comprising a second root node and a second plurality of child nodes, each child node having a label; for each child node of the first ontology, assigning at least one synthetic identifier to the child node, wherein each synthetic identifier has a length that is based on a path from the first root node to the child node of the first ontology; for each child node of the second ontology, assigning at least one synthetic identifier to the child node, wherein each synthetic identifier has a length that is based on a path from the second root node to the child node of the second ontology; pre-training a translation model at least in part by, applying one or more masked language models to the first ontology, and applying one or more masked language models to the second ontology; for each of a plurality of labels of the first ontology, identifying one or more labels of the second ontology that match the label of the first ontology, augmenting the first ontology based on the identified one or more labels of the second ontology, and augmenting the second ontology based on the label of the first ontology; fine-tuning the pre-trained translation model at least in part by training the translation model on the augmented first ontology and the augmented second ontology; receiving a translation request, the translation request include a text string and identifying a target ontology; and applying the fine-tuned translation model to the received translation request to identify at least one or more child nodes of the target ontology that correspond to the text string of the received translation request.

Example 16: The computer-readable storage medium of any of the Examples herein, the method further comprising: for each of a plurality of child nodes of the first ontology, applying the fine-tuned translation model to the child node to identify one or more child nodes of the second ontology, and for each identified one or more child nodes of the second ontology, storing a synthetic identifier assigned to the identified child node of the second ontology in association with the child node of the first ontology.

Example 17: The computer-readable storage medium of any of the Examples herein, the method further comprising: for a first child node of the first plurality of child nodes, selecting, from among a plurality of synthetic identifiers assigned to the first child node, a primary synthetic identifier based on the length of each of the plurality of synthetic identifiers assigned to the first child node.

Example 18: The computer-readable storage medium of any of the Examples herein, wherein applying the one or more masked language models to the first ontology comprises masking a first percentage of labels in a first iteration and masking a second percentage of labels in a second iteration, wherein the second percentage is greater than the first percentage.

Example 19: The computer-readable storage medium of any of the Examples herein, the method further comprising: determining a height of the first ontology; and in response to determining that the determined height is greater than a predetermined threshold, applying a clustering algorithm to the first plurality of child nodes to generate a synthetic hierarchy for the first ontology.

Example 20: The computer-readable storage medium of any of the Examples herein, the method further comprising: generating synthetic semantic identifiers for each of a plurality of cluster nodes and leaf nodes of the synthetic hierarchy.

1. A method, performed by a computing system having a memory and a processor, for aligning a first ontology of a first data source and a second ontology of a second data source, the method comprising:
receiving a specification for the first ontology, the first ontology comprising a first root node and a first plurality of child nodes, each child node having a label;
receiving a specification for the second ontology, the second ontology comprising a second root node and a second plurality of child nodes, each child node having a label;
for each child node of the first ontology, assigning at least one synthetic identifier to the child node, wherein each synthetic identifier has a length that is based on a path from the first root node to the child node of the first ontology;
for each child node of the second ontology, assigning at least one synthetic identifier to the child node, wherein each synthetic identifier has a length that is based on a path from the second root node to the child node of the second ontology;
pre-training a translation model at least in part by,
applying one or more masked language models to the first ontology, and
applying one or more masked language models to the second ontology;
for each of a plurality of labels of the first ontology,
identifying one or more labels of the second ontology that match the label of the first ontology,
augmenting the first ontology based on the identified one or more labels of the second ontology, and
augmenting the second ontology based on the label of the first ontology;
fine-tuning the pre-trained translation model at least in part by training the translation model on the augmented first ontology and the augmented second ontology;
receiving a translation request, the translation request include a text string and identifying a target ontology; and
applying the fine-tuned translation model to the received translation request to identify at least one or more child nodes of the target ontology that correspond to the text string of the received translation request.

2. The method of claim 1, further comprising:
for each of a plurality of child nodes of the first ontology,
applying the fine-tuned translation model to the child node to identify one or more child nodes of the second ontology, and
for each identified one or more child nodes of the second ontology,
storing a synthetic identifier assigned to the identified child node of the second ontology in association with the child node of the first ontology.

3. The method of claim 1, further comprising:
for a first child node of the first plurality of child nodes,
selecting, from among a plurality of synthetic identifiers assigned to the first child node, a primary synthetic identifier based on the length of each of the plurality of synthetic identifiers assigned to the first child node.

4. The method of claim 1, wherein applying the one or more masked language models to the first ontology comprises masking a first percentage of labels in a first iteration and masking a second percentage of labels in a second iteration, wherein the second percentage is greater than the first percentage.

5. The method of claim 1, further comprising:
determining a height of the first ontology; and
in response to determining that the determined height is greater than a predetermined threshold, applying a clustering algorithm to the first plurality of child nodes to generate a synthetic hierarchy for the first ontology.

6. The method of claim 5, further comprising:
generating synthetic semantic identifiers for each of a plurality of cluster nodes and leaf nodes of the synthetic hierarchy.

7. The method of claim 1, further comprising:
for each identified one or more child nodes of the second ontology,
storing a synthetic identifier assigned to the child node of the first ontology in association with the identified child node of the second ontology.

8. A computing system for aligning ontologies, the computing system comprising:
at least one processor;
at least one memory;
a component configured to receive a specification for a first ontology, the first ontology comprising a first root node and a first plurality of child nodes, each child node having a label;
a component configured to receive a specification for a second ontology, the second ontology comprising a second root node and a second plurality of child nodes, each child node having a label;
a component configured to, for each child node of the first ontology, assign at least one synthetic identifier to the child node, wherein each synthetic identifier has a length that is based on a path from the first root node to the child node of the first ontology;
a component configured to, for each child node of the second ontology, assign at least one synthetic identifier to the child node, wherein each synthetic identifier has a length that is based on a path from the second root node to the child node of the second ontology;
a component configured to, pre-train a translation model at least in part by,
applying one or more masked language models to the first ontology, and
applying one or more masked language models to the second ontology;
a component configured to, for each of a plurality of labels of the first ontology,
identify one or more labels of the second ontology that match the label of the first ontology,
augment the first ontology based on the identified one or more labels of the second ontology, and
augment the second ontology based on the label of the first ontology;
a component configured to fine-tune the pre-trained translation model at least in part by training the translation model on the augmented first ontology and the augmented second ontology;
a component configured to receive a translation request, the translation request include a text string and identifying a target ontology; and
a component configured to apply the fine-tuned translation model to the received translation request to identify at least one or more child nodes of the target ontology that correspond to the text string of the received translation request,
wherein each component comprises computer-executable instructions stored in the at least one memory for execution by the computing system.

9. The computing system of claim 8, further comprising:
a component configured to, for each of a plurality of child nodes of the first ontology,
apply the fine-tuned translation model to the child node to identify one or more child nodes of the second ontology, and
for each identified one or more child nodes of the second ontology,
store a synthetic identifier assigned to the identified child node of the second ontology in association with the child node of the first ontology.

10. The computing system of claim 8, further comprising:
a component configured to, for a first child node of the first plurality of child nodes,
select, from among a plurality of synthetic identifiers assigned to the first child node, a primary synthetic identifier based on the length of each of the plurality of synthetic identifiers assigned to the first child node.

11. The computing system of claim 8, wherein the component configured to apply the one or more masked language models to the first ontology is further configured to mask a first percentage of labels in a first iteration and mask a second percentage of labels in a second iteration, wherein the second percentage is greater than the first percentage.

12. The computing system of claim 8, further comprising:
a component configured to determine a height of the first ontology; and
a component configured to, in response to determining that the determined height is greater than a predetermined threshold, apply a clustering algorithm to the first plurality of child nodes to generate a synthetic hierarchy for the first ontology.

13. The computing system of claim 12, further comprising:
a component configured to generate synthetic semantic identifiers for each of a plurality of cluster nodes and leaf nodes of the synthetic hierarchy.

14. The computing system of claim 8, further comprising:
a component configured to, for each identified one or more child nodes of the second ontology,
store a synthetic identifier assigned to the child node of the first ontology in association with the identified child node of the second ontology.

15. A computer-readable storage medium storing instructions that, when executed by a computing system having a memory and a processor, cause the computing system to perform a method for aligning a plurality of ontologies, the method comprising:
receiving a specification for a first ontology, the first ontology comprising a first root node and a first plurality of child nodes, each child node having a label;
receiving a specification for a second ontology, the second ontology comprising a second root node and a second plurality of child nodes, each child node having a label;
for each child node of the first ontology, assigning at least one synthetic identifier to the child node, wherein each synthetic identifier has a length that is based on a path from the first root node to the child node of the first ontology;
for each child node of the second ontology, assigning at least one synthetic identifier to the child node, wherein each synthetic identifier has a length that is based on a path from the second root node to the child node of the second ontology;
pre-training a translation model at least in part by,
applying one or more masked language models to the first ontology, and
applying one or more masked language models to the second ontology;
for each of a plurality of labels of the first ontology,
identifying one or more labels of the second ontology that match the label of the first ontology,
augmenting the first ontology based on the identified one or more labels of the second ontology, and
augmenting the second ontology based on the label of the first ontology;
fine-tuning the pre-trained translation model at least in part by training the translation model on the augmented first ontology and the augmented second ontology;
receiving a translation request, the translation request include a text string and identifying a target ontology; and
applying the fine-tuned translation model to the received translation request to identify at least one or more child nodes of the target ontology that correspond to the text string of the received translation request.

16. The computer-readable storage medium of claim 15, the method further comprising:
for each of a plurality of child nodes of the first ontology,
applying the fine-tuned translation model to the child node to identify one or more child nodes of the second ontology, and
for each identified one or more child nodes of the second ontology,
storing a synthetic identifier assigned to the identified child node of the second ontology in association with the child node of the first ontology.

17. The computer-readable storage medium of claim 15, the method further comprising:
for a first child node of the first plurality of child nodes,
selecting, from among a plurality of synthetic identifiers assigned to the first child node, a primary synthetic identifier based on the length of each of the plurality of synthetic identifiers assigned to the first child node.

18. The computer-readable storage medium of claim 15, wherein applying the one or more masked language models to the first ontology comprises masking a first percentage of labels in a first iteration and masking a second percentage of labels in a second iteration, wherein the second percentage is greater than the first percentage.

19. The computer-readable storage medium of claim 15, the method further comprising:
determining a height of the first ontology; and
in response to determining that the determined height is greater than a predetermined threshold, applying a clustering algorithm to the first plurality of child nodes to generate a synthetic hierarchy for the first ontology.

20. The computer-readable storage medium of claim 19, the method further comprising:
generating synthetic semantic identifiers for each of a plurality of cluster nodes and leaf nodes of the synthetic hierarchy.
